(19) United States
(12) Patent Application Publication
(10) Pub. No.: US 2026/0331279 A1
(43) Pub. Date: Oct. 22, 2026

(54) DYNAMICALLY SELECTING CONTEXT FOR DATA DISPLAYED TO A USER BASED ON SENTIMENT ANALYSIS OF USER INTERACTION WITH THE DISPLAYED DATA

(71) Applicant: Ethicon Endo-Surgery, Inc., Cincinnati, OH (US)

(72) Inventor: Manikandan Rajappa, North Wales, PA (US)

(21) Appl. No.: 19/641,101

(22) Filed: Apr. 7, 2026

Related U.S. Application Data
(60) Provisional application No. 63/789,619, filed on Apr. 16, 2025.

(51) Int. Cl. G16H 40/67; G06F 3/01; G06F 3/14; G06V 20/40; G06V 40/16; G06V 40/18; G06V 40/20

(52) U.S. Cl. CPC G16H 40/67; G06F 3/012; G06F 3/013; G06F 3/14; G06V 20/41; G06V 40/166; G06V 40/171; G06V 40/193; G06V 40/20

(57) ABSTRACT

A client device displays data to a user through an application associated with an online medical system. The application captures interaction with the data by the user, which includes video of the user’s face captured by an image capture device of the client device. The online medical system receives the video data and determines a sentiment of the user towards the data based on attributes of the user’s face. A reading behavior of the user for the data may also be determined from tracking the user’s gaze in the video. Based on the sentiment, the online medical system determines whether to present supplemental content to the user. In response to determining to present supplemental content, the online medical system determines the supplemental content based on the determined sentiment and the data displayed by the client device.

### RELATED APPLICATIONS

This application claims the benefit of U.S. Provisional Application No. 63/789,619 filed on Apr. 16, 2025, which is incorporated by reference herein.

### BACKGROUND

### Technical Field

The described embodiments relate to augmenting data displayed by a client device to a user with supplemental information selected based on user interaction with data displayed by the client device.

### Description of the Related Art

Patients are often presented with a variety of documents and literature relating to one or more medical procedures. For example, a patient is presented with various literature describing a medical procedure as part of instructions for preparing for the medical procedure. Similarly, a patient is presented with various literature describing steps for recovering from the medical procedure. Additional descriptive information about a medical procedure, a medical facility, medications, or a medical practitioner may be available for a patient to review as well.

Patients often review data, such as documents or literature, using client devices such as mobile devices or laptop computers. Additionally, data reviewed by a patient may include portions that are unclear or confusing to the patient. Conventionally, a patient requests additional information about portions of data that are unclear by communicating with a medical facility or a medical practitioner associated with the data. For example, a patient transmits a message to a medical facility including one or more questions to a medical facility or a to medical procedure to obtain additional clarifying information. This increases an amount of time for a patient to understand certain data and increases an amount of data exchanged between a patient and a medical facility, increasing an amount of bandwidth used by a medical facility to exchange data with various patients.

### BRIEF DESCRIPTION OF THE DRAWINGS

FIG. 1 is an example embodiment of a computing environment in which an online medical system operates.

FIG. 2 is an interaction diagram of an example embodiment of a method for selecting supplemental content for a user based on interactions with data captured by a client device.

FIG. 3 is a process flow diagram of an example embodiment of a method for selecting supplemental content for a user based on interactions with data captured by the client device.

### DETAILED DESCRIPTION

The Figures(FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

A client device displays data to a user through an application associated with an online medical system. The data may be obtained from the online medical system or from a third-party system in various embodiments. As the user views the displayed data, the application captures interaction with the data by the user through the client device. The captured interaction includes video of the user’s face while viewing the displayed data captured by an image capture device of the client device. Other information, such as inputs received by the client device to navigate through portions of the data, may be included in the captured interaction in various embodiments. The application transmits the captured interaction, including the captured video of the user’s face, along with an identifier of the displayed data to the online medical system.

Based on the received video data of the user’s face, the online medical system determines a sentiment of the user towards the data based on attributes of the user’s face in the video data. For example, the online medical system applies one or more sentiment analysis models to the video data to determine the sentiment of the user. In some embodiments, the online medical system also applies one or more gaze tracking models to the received video data to track the user’s gaze and to determine a reading behavior of the user for the data from the user’s gaze in the video data. Based on the sentiment, the online medical system determines whether to present supplemental content to the user. For example, in response to the sentiment satisfying one or more criteria, the online medical system detemrines to present supplemental content to the user. Similarly, the online medical system may determine to present supplemental content to the user in response to the determined reading behavior satisfying one or more criteria.

In response to determining to present supplemental content, the online medical system determines the supplemental content based on the determined sentiment and the data displayed by the client device. For example, the online medical system maintains associations between different supplemental content and combinations of identifiers of data and sentiments. In the preceding example, the online medical system selects supplemental content associated with the combination of the identifier of data displayed by the client device and determined sentiment. Alternatively, the online medical system applies a generative model to the identifier of the displayed data (or to the displayed data) and the determined sentiment, with the generative model determining the supplemental content. The online medical system transmits the supplemental content to the client device for presentation. In various embodiments, the client device displays the supplemental content as a prompt overlaid on a portion of the displayed content.

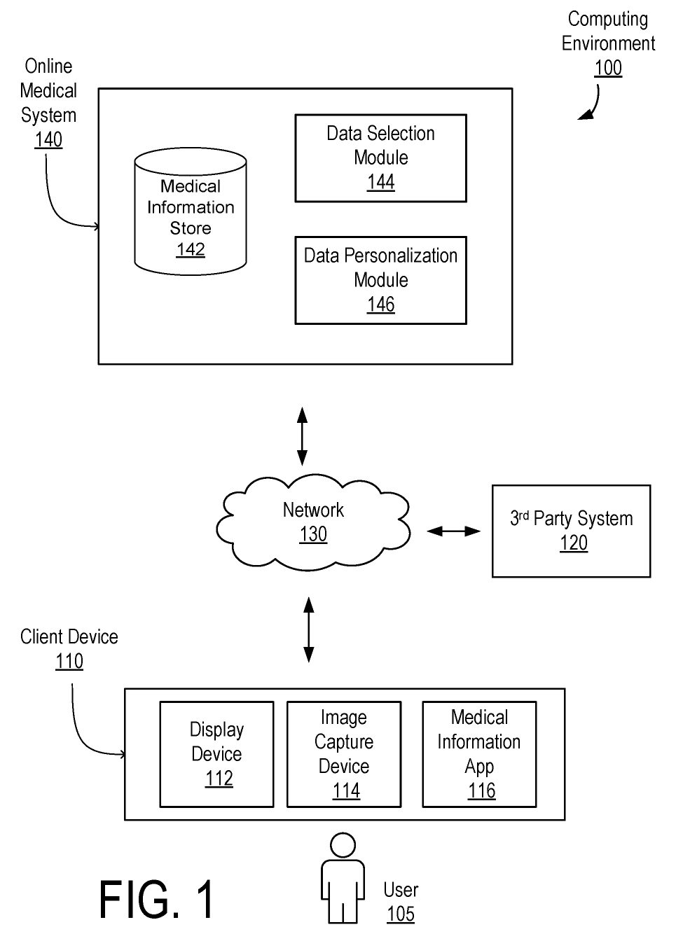

FIG. 1 illustrates an example embodiment of a computing environment 100 in which an online medical system 140 operates. The online medical system 140 may include one or more servers that are coupled by a network 130 to client devices 110 associated with users 105 of the online medical system 140, and to various third-party systems 120. The online medical system 140 facilitates exchange of information between one or more users 105 and medical practitioners, medical facilities, administrators, or other users 105 via client devices 110. For example, the online medical system 140 provides information about one or more medical procedures to a client device 110 for presentation to a user. Additionally, the online medical system 140 may facilitate access to various third-party systems 120 providing external services for a user accessible via a client device 110. Example external services provided by a third-party system 120 include electronic healthcare records (EHR) services, medical telepresence services, operating room scheduling, data analytics services, etc.

The online medical system 140 may be implemented using on-site computing or storage systems, cloud computing or storage systems, or a combination thereof. The online medical system 140 may be implemented using local or cloud-based servers, which may include physical or virtual machines, or a combination thereof. Cloud-based servers may include private cloud systems, public cloud systems, hybrid public/private cloud systems, or a combination thereof. Accordingly, the online medical system 140 may be local, remote, and/or distributed relative to the medical environments where medical procedures are performed and relative to one or more client devices 110 providing user access. Furthermore, different portions of the online medical system 140 may execute on different remote servers and various system elements of the online medical system 140 may be communicatively coupled over a network 130.

Examples of the client device 110 include: a mobile phone, a tablet, a laptop computer, or a desktop computer, other computing device, or application executing thereon for accessing the online medical system 140 via the network 130. The client device 110 may enable access to various user interfaces (which may comprise web-based interfaces accessed via a browser or application interfaces accessed via an application) for viewing or editing information associated with the online medical system 140. The client device 110 may include conventional computer hardware such as a display device, an input device (e.g., touch screen), a memory, a processor, and a non-transitory computer-readable storage medium that stores instructions for execution by the processor to carry out functions described herein.

For purposes of illustration, FIG. 1 shows an example embodiment where the client device 110 includes a display device 112, an image capture device 114, and a medical information application 116. However, the client device 110 includes additional components than those illustrated in FIG. 1 in various embodiments. The display device 112 displays data to the user 105. For example, the display device 112 displays data the client device 110 obtains from the online medical system 140, from a third-party system 120, or from another device. As another example, the display device 112 displays data generated by the client device 110. The display device 112 is integrated into the client device 110 in some embodiments, while in other embodiments the display device 112 is coupled to the client device 110.

The client device 110 also includes one or more image capture devices 114. An image capture device 114 captures images or video of an area including the client device 110 that is within a field of view of the image capture device 114. In various embodiments, an image capture device 114 is positioned to have a field of view including a user 105 viewing the display device 112. The client device 110 includes multiple image capture devices 114 in some embodiments, with different image capture devices 114 having different fields of view capturing different portions of an area including the client device 110. For example, an image capture device 114 has a field of view including a portion of the area including the client device 110 that the display device 112 faces, while another image capture device 114 has a field of view including a portion of the area including the client device 110 that is opposite to a direction that the display device 112 faces.

Additionally, a medical information application 116 executes on the client device 110 to exchange data with the online medical system 140 via the network 130. The medical information application 116 obtains data from the online medical system 140 and presents the obtained data to a user 105. For example, the medical information application 116 generates one or more interfaces displaying data relating to a medical procedure based on information associated with the medical procedure by the online medical system 140. Other information may be included in an interface generated by the medical information application 116 in various embodiments. Similarly, the medical information application 116 transmits data from the client device 110 to the online medical system 140.

In various embodiments, the medical information application 116 transmits one or more control signals to one or more devices of the client device 110. A control signal may activate or deactivate a device of the client device 110. For example, the medical information application 116 transmits a control signal to the image capture device 114 to activate the image capture device 114 for capturing video or images that are received by the medical information application 116. Similarly, the medical information application 116 transmits an alternative control signal to the image capture device 114 to deactivate the image capture device 114, which stops capture of images or video by the image capture device 114. In various embodiments, the user 105 expressly authorizes the medical information application 116 to transmit one or more control signals to one or more devices of the client device 110. For example, the client device 110 stores authentication information from the user 105 for the medical information application 116 to transmit one or more control signals to a device included in the client device 110 or for the medical information application 116 to access information captured by one or more of the devices included in the client device 110. Such authentication information may include an identifier of a device and data describing control signals that the medical information application 116 is authorized by the user to transmit to the device corresponding to the identifier or identifying data from a device the medical information application 116 is authorized to access.

In various embodiments, the medical information application 116 expressly prompts the user 105 for permission to access data captured by one or more devices of the client device 110. The medical information application 116 does not access data captured by a device unless the client device 110 maintains authorization from the user for the medical information application 116 to access data captured by the device in various embodiments, so the user 105 regulates access to data obtained by one or more devices (e.g., an image capture device 114) of the client device 110 or to regulate access to one or more devices (e.g., an image capture device 114) of the client device 110 by the medical information application 116. Alternatively, the medical information application 116 is authorized to access one or more devices (e.g., an image capture device 114) of the client device 110, and has one or more settings or options the user 105 may select or modify to prevent the medical information application 116 from accessing one or more devices, such as the image capture device 114, of the client device 110 corresponding to one or more of the settings.

Additionally, the medical information application 116 captures interaction by the user 105 with data displayed by the medical information application 116. The captured interaction includes video of the user 105 captured by an image capture device 114 while the user views the data displayed by the client device 110. For example, the medical information application 116 activates an image capture device 114 having a field of view including a portion of an area including the client device 110 that the display device 112 of the client device 110 faces. Additional information may be included in the captured interaction with data displayed via the client device 110 in some embodiments. For example, the medical information application 116 captures one or more gestures or other inputs received by one or more input devices of the client device 110. Example input devices include a touchscreen, a microphone, an accelerometer, or other sensors capable of receiving one or more inputs from a user. Example inputs or gestures included in the interaction include inputs for navigating through the displayed data (e.g., scrolling inputs, scrolling gestures, etc.) or for identifying different portions of the displayed data.

The medical information application 116 transmits the captured interactions by the user 105 in association with information identifying data displayed by the medical information application 116 to the online medical system 140. As further described below, the online medical system 140 determines a sentiment of the user 105 based on the captured interactions and selects supplemental content for presentation to the user 105 based on the determined sentiment. For example, the online medical system 140 maintains one or more rules associating different supplemental content with different combinations of identifiers of data displayed by the medical information application 116 and sentiments. The online medical system 140 transmits supplemental content associated with a combination of identifier of data displayed by the medical information application 116 and a determined sentiment for the user to the medical information application 116. Subsequently, the medical information application 116 displays the supplemental content to the user 105.

In some embodiments, the supplemental content the medical information application 116 receives from the online medical system 140 comprises an interactive prompt capable of receiving input, such as text data, from the user 105. The medical information application 116 transmits the received text to the online medical system 140, which applies a trained large language model (LLM) to the received text to generate additional content that is transmitted to the client device 110 and presented to the user 105 via the medical information application 116. For example, the interactive prompt presents the additional content received from the online medical system 140. Hence, supplemental content from the online medical system 140 allows the user 105 to more easily interact with the online medical system 140 to obtain additional content based on supplemental content from the online medical system 140.

A third-party system 120 may facilitate diverse services used by the online medical system 140. While FIG. 1 shows a single third-party system 120, in various embodiments multiple third-party systems 120 are included in the computing environment 100. For example, the third-party system 120 may include various electronic health record (her) systems for managing patient records, telepresence servers for facilitating telepresence services, patient scheduling systems, hospital information systems (HIS), or other servers. As another example, one or more third-party systems 120 include educational content about various medical procedures, such as articles about various medical procedures, audio data related to medical procedures, video data related to medical procedures, or other descriptive information about medical procedures. The third-party system 120 may be implemented using various on-site computing or storage systems, cloud computing or storage systems such as private cloud systems, public cloud systems, hybrid public/private cloud systems, or a combination thereof.

The network 130 comprises communication pathways for communication between one or more client devices 110, one or more third-party systems 120, and the online medical system 140. The network 130 may include one or more local area networks and/or one or more wide area networks (including the Internet). The network 130 may also include one or more direct wired or wireless connections (e.g., Ethernet, WiFi, cellular protocols, WiFi direct, Bluetooth, Universal Serial Bus (USB), or other communication link).

The online medical system 140 maintains information associated with one or more medical procedures and transmits information associated with a medical procedure to a client device 110 for presentation to a user 105. Example information associated with a medical procedure includes: instructions for preparing for the medical procedure, instructions for recovering from the medical procedure, descriptive information about the medical procedure, information about one or more medications relating to the medical procedure, information about one or more anatomical structures, or other types of information relating to one or more medical procedures. However, the online medical system 140 maintains additional or alternative information, such as information about the user 105 (e.g., medical records of the user 105, contact information about the user 105), information about one or more medical practitioners, information about a medical facility, or other information accessible to the user 105.

In the example shown by FIG. 1, the online medical system 140 includes a medical information store 142, a data selection module 144, and a data personalization module 146. However, in other embodiments, the online medical system 140 includes different or additional components than those depicted by FIG. 1. Further, in some embodiments, a single component may provide the functionality of multiple components shown by FIG. 1; for example, a single component provides the functionality of the data selection module 144 and the data personalization module 146, further described below.

The medical information store 142 includes information associated with one or more medical procedures. For example, the medical information store 142 maintains an identifier for each of a set of medical procedures and associates data with each identifier. Data associated with an identifier of a medical procedure includes text data, video data, audio data, or a combination thereof. Example information associated with a medical procedure includes: text data about the medical procedure, video data about the medical procedure, audio data about the medical procedure, or other types of information describing the medical procedure. Different data may be associated with different identifiers, allowing different data to be maintained for different medical procedures. Data associated with an identifier of a medical procedure may include one or more forms for receiving information from a user 105 or from another entity. Further, the medical information store 142 may associate data with an identifier of a user 105. For example, the medical information store 142 maintains associations between one or more medical procedures and an identifier of a user 105 to identify medical procedures relevant to the user 105 (e.g., medical procedures scheduled for the user 105, medical procedures for which the user 105 previously requested information, etc.).

The data selection module 144 selects one or more portions of data from the medical information store 142 for transmitting to a client device 110 for presentation to the user 105. In various embodiments, the data selection module 144 receives a request for data from the medical information application 116 including an identifier of a medical procedure. For example, the data selection module 144 retrieves at least a subset of data associated with the received identifier of the medical procedure and transmits the retrieved subset of data to the medical information application 116 for presentation. As another example, the data selection module 144 receives a request including an identifier of the user 105 and selects data based on the identifier of the user 105. For example, the data selection module 144 determines one or more medical procedures associated with the identifier of the user included in the request from the medical information store 142 and retrieves data associated with one or more of the medical procedures associated with the identifier of the user 105 from the medical information store 142. As another example, the data selection module 144 receives a request including a search query and retrieves data included in the medical information store 142 including data or metadata that at least partially matches the search query. The data selection module 144 transmits the data selected in response to a request to the client device 110 from which the request was received for presentation.

The data personalization module 146 selects supplemental content for augmenting data selected by the data selection module 144 based on interaction by the user 105 with the data displayed by the client device 110 that was captured by the client device 110. As further described above, the medical information application 116 captures interactions with data displayed by the client device 110 and transmits the captured interaction to the data personalization module 146 in association with an identifier of the data displayed by the client device 110. Based on the captured interaction, the data personalization module 146 determines a sentiment of the user 105 regarding the data displayed by the client device 110. The data personalization module 146 selects supplemental content for the user 105 based on the sentiment to complement the data displayed by the client device 110. Supplemental content may provide contextual information clarifying or explaining a portion of the data presented by the client device 110. Alternatively or additionally, supplemental content provides instructions or suggestions for reviewing the data displayed by the client device 110, such as suggestions to pause reviewing the data displayed by the client device 110 or suggestions for alternative data for the user 105 to review. Supplemental content may also comprise suggestions for additional data related to the data presented by the client device 110 or other types of content in various embodiments.

In various embodiments, the data personalization module 146 applies one or more machine learning models to the interaction received from the medical information application 116. The interaction includes video of a face of a user 105 viewing the data displayed by the client device 110, and the data personalization module 146 applies one or more trained sentiment analysis models to the face of the user 105 in the video, with a sentiment analysis model determining a sentiment of the user 105 based on the video. Examples of sentiments include: confusion, frustration, interest, anger, or other indications of the user’s reaction to the data presented by the client device 110. In various embodiments, a sentiment analysis model detects the user’s face in the video from the client device 110 and determines the user’s sentiment based on attributes of the user’s face in the video. The data personalization module 146 determines the sentiment of the user 105 at different times in the video, identifying changes in the user’s sentiment over time while viewing the data displayed by the client device 110.

The data personalization module 146 determines whether to obtain supplemental content for the user 105 based on the determined sentiment for the user 105. In various embodiments, the data personalization module 146 determines whether a sentiment determined for the user 105 satisfies one or more criteria. For example, the data personalization module 146 determines whether a determined sentiment is included in a set of sentiments. In response to determining the sentiment determined for the user is included in the set of sentiments, the data personalization module 146 selects supplemental content for presentation to the user 105. For example, the set of sentiments comprises: frustration, confusion, and interest, so the data personalization module 146 selects supplemental content for the user 105 in response to a determined sentiment for the user 105 being frustration, confusion, or interest. However, the data personalization module 146 does not obtain supplemental content for the user 105 in response to determining a sentiment for the user 105 that is not included in the set of sentiments (or that does not otherwise satisfy one or more criteria).

In some embodiments, the data personalization module 146 also analyzes eye movement of the user 105 based on the received video to evaluate a reading behavior describing focus, attention, or engagement of the user 105 with the data displayed by the client device 110. For example, the data personalization module 146 leverages one or more gaze tracking methods to determine eye movement patterns of the user 105 while viewing the data presented by the client device 110. Different eye movement patterns may be determined by one or more gaze tracking methods in various embodiments. Based on one or more eye movement patterns, the data personalization module 146 determines a reading behavior of the user 105 with the data presented by the client device 110. For example, based on changes in gaze of the user’s eyes over time, the data personalization module 146 determines a duration that the user’s gaze is fixed on one or more portions of the data presented by the client device 110, determines whether the user’s gaze returns to a portion of the data presented by the client device 110 multiple times (indicating the user is re-reading the portion of the data), determines whether the user has lost focus in the data displayed by the client device 110 with different determinations corresponding to different reading behaviors. In various embodiments, data from a gaze tracking model describing the user’s gaze is input into a trained classification model that determines the reading behavior of the user 105 based on gaze data.

In various embodiments, the data personalization module 146 determines whether to obtain supplemental content based on a combination of the sentiment and the reading behavior determined for the user 105. In various embodiments, the data personalization module 146 determines whether a sentiment determined for the user 105 satisfies one or more sentiment criteria and determines whether a reading behavior of the user 105 satisfies one or more reading criteria. For example, the data personalization module 146 determines whether a sentiment determined for the user is included in a set of sentiments and determines whether a reading behavior determined for the user is included in a set of reading behaviors. The set of reading behaviors includes one or more of: the user’s gaze being fixed on a portions of the data presented by the client device 110 for at least a threshold amount of time, the user’s gaze returning to a portion of the data presented by the client device 110 at least a threshold number of times, and an indication the user 105 has lost focus in the data displayed by the client device 110. In other embodiments, the set of reading behaviors includes different or additional reading behaviors. In response to determining the sentiment determined for the user 105 is included in the set of sentiments or in response to determining the reading behavior determined for the user 105 is included in the set of reading behaviors, the data personalization module 146 selects supplemental content. This obtains supplemental content for the data presented by the client device 110 when the sentiment determined for the user 105 or the reading behavior determined for the user 105 satisfies one or more sentiment criteria or satisfies one or more reading criteria, respectively.

The data personalization module 146 obtains supplemental content for presentation to the user 105 based on the sentiment or the reading behavior determined for the user 105. In various embodiments, the sentiment determined for the user 105 determines the supplemental content selected by the data personalization module 146. For example, in response to determining a sentiment of confusion for the user 105, the data personalization module 146 selects supplemental content clarifying, defining, or explaining a portion of the data presented by the client device 110. In various embodiments, the data personalization module 146 determines the portion of the data presented by the client device 110 corresponding to the determined sentiment based on gaze tracking of the user’s gaze and selects supplemental content explaining, defining, or clarifying the determined portion of the data presented by the client device 110. As another example, in response to determining a sentiment of frustration for the user 105, the data personalization module 146 selects supplemental content comprising a message or a notification for the user 105 to pause viewing the data presented by the client device 110 or selects supplemental content comprising identifying alternative data for the user 105 similar to the displayed data (e.g., simplified data corresponding to the data presented by the client device 110). In another example, in response to determining a sentiment of interest for the user 105, the data personalization module 146 selects supplemental content identifying additional data related to the data presented by the client device 110, such as data providing further information about one or more portions of the data presented by the client device 110. Other types of supplemental content may be selected in response to determining other sentiments for the user 105.

In various embodiments, the data personalization module 146 selects supplemental content based on the data presented by the client device 110 and the sentiment determined for the user, and also accounts for the reading behavior of the user 105 in some embodiments. For example, the data personalization module 146 stores associations between different portions of supplemental content and combinations of sentiments and identifiers of data (e.g., an identifier of the data displayed by the client device 110, an identifier of a portion of the data displayed by the client device 110, etc.). The combination may additionally include a reading behavior in various embodiments. The data personalization module 146 selects a portion of supplemental content associated with a combination of a determined sentiment for the user 105 and an identifier of data presented by the client device 110. In various embodiments, the data personalization module 146 determines a portion of the data presented by the client device 110 based on tracking the user’s gaze to select supplemental content tailored to specific portion of the data presented by the client device 110. Supplemental content may be text data, video data, audio data, or a combination thereof in various embodiments. For example, supplemental content includes one or more text articles describing or explaining a portion of the data displayed by the client device 110 or one or more videos explaining one or more portions of the data displayed by the client device 110. As another example, supplemental content includes one or more text articles having different phrasing or presentation of portions of the data displayed by the client device 110 or video data having content that provides further details about one or more portions of the data displayed by the client device 110.

In some embodiments, the data personalization module 146 applies a trained generative model, such as a large language model (LLM) to the determined sentiment for the user 105 and to the identifier of the data presented by the client device 110 (as well as a to reading behavior determined for the user 105 in some embodiments). The generative model is a generative pre-trained transformer (GPT) model in various embodiments. For example, the data personalization module 146 generates a prompt including the determined sentiment for the user 105 and the identifier of the portion of the data presented by the client device 110 (and including a reading behavior determined for the user 105 in some embodiments). The prompt also includes an instruction to obtain supplemental content for the user 105 based on the determined sentiment. The generative model may generate text in response to a text prompt in various embodiments. Alternatively or additionally, the generative model selects video data or audio data in response to a text prompt. In various embodiments, the generative model is pre-trained on a text corpus including text to output text in response to a text prompt from a user. The data personalization module 146 obtains and applies a pre-trained generative model in various embodiments to leverage relationships between different text (or other types of data) the generative model learned through application to a text corpus or other corpus including a larger amount of data and more varied data than maintained by the online medical system 140. Based on the prompt and relationships learned during a pre-training process, the generative model selects supplemental content, which may be content generated by the generative model or identifiers of portions of content determined by the generative model.

The data personalization module 146 transmits the selected supplemental content to the client device 110 for presentation to the user. In various embodiments, the supplemental content comprises a prompt displayed in conjunction with the data displayed by the client device 110. For example, the prompt including the supplemental content is overlaid on a portion of the display device 112, while the remaining portions of the display device 112 present the data previously displayed by the client device 110. In response to one or more interactions with the prompt, the client device 110 retrieves additional data identified by the prompt from the online medical system 140 or from a third-party system 120 and updates the display device 112 to display the additional data.

Further, the supplemental content may comprise an interactive prompt that is presented to the user 105 via the client device. The interactive prompt displays content to the user 105, and also receives input from the user 105, such as text data. Input received via the interactive prompt is received by the data personalization module 146, which applies a generative model (e.g., an LLM) to the received input to generate additional content that is presented to the user via the interactive prompt. This allows the data personalization module 146 to dynamically modify the supplemental content in response to received input from the user. Hence, the interactive prompt may present supplemental content to the user 105 that the data personalization module 146 generated in response to input received from the user 105 in various embodiments.

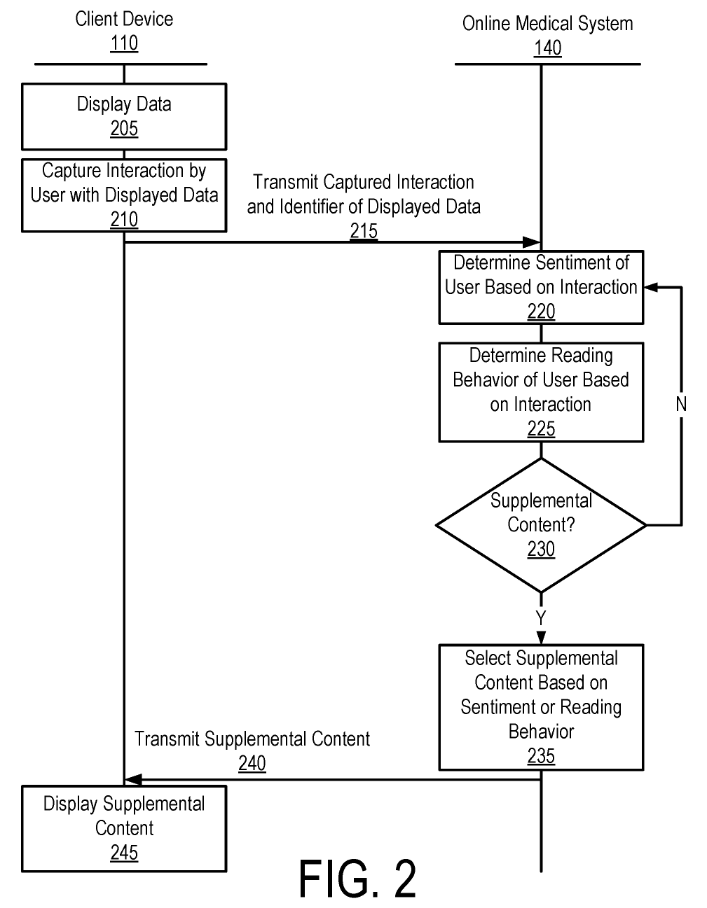

FIG. 2 is an interaction diagram of an example embodiment of a method for selecting supplemental content for a user based on interaction with data captured by a client device 110. A client device 110 displays 205 data from an online medical system 140 (or from a third-party system 120) to a user 105 via a display device 112. For example, the data comprises text or other content associated with a medical procedure. Example content associated with a medical procedure includes: a description of the medical procedure, instructions for preparing for the medical procedure, instructions for recovering from the medical procedure, forms for obtaining information from the user 105 relevant to the medical procedure, or other data relevant to a medical procedure. A medical information application 116 associated with the online medical system 140 obtains the data and displays 205 the data via the display device 112. However, other data, such as data unrelated to a medical procedure, may be displayed 205 in various embodiments. Further, data displayed 205 by the client device 110 may be obtained from a third-party system 120 and displayed 205 via the medical information application 116 in some embodiments.

While the client device 110 displays 205 the data, the medical information application 116 captures 210 interaction by the user with the displayed data. In various embodiments, the medical information application 116 activates an image capture device 114 of the client device 110 and obtains video of a face of the user 105 while viewing the data via the client device 110 as interaction with the displayed data. Additional information may be captured 210 as interaction with the data in various embodiments. For example, interaction includes received inputs to navigate the displayed data received by the client device 110, such as scrolling through the displayed data or other inputs from the user 105 navigating through portions of the displayed data.

The client device 110 transmits 215 the captured interactions and an identifier of the displayed data to the online medical system 140. For example, the identifier comprises a name or another unique identifier of the data displayed 205 by the client device 110. The captured interaction includes the video of the user’s face, and may include descriptions of other inputs received from the user to navigate through the displayed data. The descriptions of inputs for navigating through portions of the displayed data are transmitted 215 in conjunction with the captured video of the user’s face in various embodiments.

As further described above in conjunction with FIG. 1, the online medical system 140 determines 220 a sentiment of the user based on the captured interaction. The captured interaction includes video of a face of a user 105 viewing the data displayed 205 by the client device 110, and the online medical system 140 applies one or more trained sentiment analysis models to the video of the face of the user 105. A sentiment analysis model determines 220 a sentiment of the user 105 based on features of the user’s face in the video. Examples of sentiments include: confusion, frustration, interest, anger, or other indications of the user’s reaction to the data displayed 205 by the client device 110. In some embodiments, the online medical system 140 locally determines 220 the sentiment of the user from the interaction, while in other embodiments the online medical system 140 transmits at least a subset of the captured interaction (e.g., video data of a face of the user) to a third-party system 120 that applies a sentiment analysis model to the subset of the captured interaction and transmits a determined sentiment to the online medical system 140.

In various embodiments, the online medical system 140 determines 225 a reading behavior of the user based on the interaction. For example, the online medical system 140 determines 225 a reading behavior of the user by tracking the user’s gaze directed towards the data displayed 205 by the client device 110 in the video of the user’s face. For example, the online medical system 140 applies a gaze tracking model to captured video of the user’s face received from the client device 110; data describing the user’s gaze (e.g., movement of the user’s gaze, a duration the user’s gaze is fixated on a portion of the data, etc.) is input into a trained classification model that determines 225 the reading behavior of the user 105. Based on changes in the gaze of the user’s eyes over time, the online medical system 140 determines one or more reading behaviors of the user 105. Example reading behaviors include: a duration that the user’s gaze is fixed on one or more portions of the data displayed 205 by the client device 110, a number of times the user’s gaze returns to a portion of the data displayed 205 by the client device 110 (indicating the user is re-reading the portion of the data), or an indication the user has lost focus in the data displayed 205 by the client device 110. Other reading behaviors may be determined 225 in various embodiments.

The online medical system 140 determines 230 whether to present supplemental content to the user based on the determined sentiment, based on the determined reading behavior, or based on a combination of the sentiment and the reading behavior determined for the user 105. In various embodiments, the online medical system 140 determines whether a sentiment determined 220 for the user 105 satisfies one or more sentiment criteria or whether a reading behavior determined 225 for the user 105 satisfies one or more reading criteria. In response to the sentiment satisfying a sentiment criterion or in response to determining the reading behavior satisfies a reading criterion, the online medical system 140 determines 230 to present supplemental content to the user 105. For example, a sentiment satisfies one or more sentiment criteria in response to the sentiment being included in a set of sentiments. An example set of sentiments includes confusion, frustration, and interest. Similarly, a reading behavior satisfying one or more reading criteria comprises the reading behavior being included in a set of reading behaviors. An example set of reading behaviors includes: the user’s gaze being fixed on a portion of the data displayed 205 by the client device 110 for at least a threshold amount of time, the user’s gaze returning to a portion of the data displayed 205 by the client device 110 at least a threshold number of times, and an indication the user has lost focus in the data displayed 205 by the client device 110. Alternatively, the online medical system 140 determines 230 to present supplemental content to the user in response to the sentiment satisfying a sentiment criterion and in response to determining the reading behavior satisfies a reading criterion.

In response to determining 230 to present supplemental content to the user 105, the online medical system 140 selects 235 supplemental content based on the sentiment or the reading behavior, as well as the data displayed 205 by the client device 110. Supplemental content may be text data, video data, audio data, or a combination thereof, as further described above in conjunction with FIG. 1. In various embodiments, the sentiment determined 220 for the user determines the supplemental content selected 235 by the online medical system 140. For example, in response to the determined sentiment being interest, the online medical system 140 selects 235 supplemental content comprising text data or video data associated with additional data related to at least a portion of the data displayed 205 by the client device 110. In various embodiments, the online medical system 140 determines a portion of the data presented by the client device 110 for which supplemental content is selected 235 based on gaze tracking of the user’s gaze and selects 235 supplemental content associated with the determined portion of the data presented by the client device 110. As another example, in response to the determined sentiment being frustration, the online medical system 140 selects 235 supplemental content comprising a message or a notification for the user 105 to pause viewing the content (i.e., to take a break from viewing the data presented by the client device 110) or selects 235 alternative data for the user 105 corresponding to the data displayed 205 by the client device 110 (e.g., simplified data based on the data presented by the client device 110). In another example, in response to the determined sentiment being frustration, the online medical system 140 selects supplemental content that provides clarification or explanation of one or more portions of the data displayed 205 by the client device 110, such as data using alternative language or formatting to convey information included in one or more portions of the data displayed 205 by the client device 110.

In various embodiments, the online medical system 140 accounts for a determined reading behavior of the user when selecting 235 supplemental content in some embodiments. As further described above in conjunction with FIG. 2, the online medical system 140 stores associations between different portions of supplemental content and different combinations of sentiments and identifiers of data (e.g., identifiers of the data displayed 205 by the client device 110, identifiers of a portion of the data displayed 205 by the client device 110 where the user’s gaze is directed, etc.). The online medical system 140 selects a portion of supplemental content associated with a combination of a determined sentiment for the user 105 and an identifier of data displayed 205 by the client device 110. In various embodiments, the online medical system 140 determines a portion of the displayed data based on the gaze of the user from the captured interaction and selects 235 supplemental content associated with the identifier for the determined portion of the data and with the determined sentiment. The combinations include a reading behavior in some embodiments, so the online medical system 140 selects 235 a portion of supplemental content associated with a combination including the identifier for the determined portion of the data, the determined sentiment, and the determined reading behavior.

In some embodiments, the online medical system 140 applies a trained generative model, such as a large language model (LLM) to the determined sentiment and to the identifier of the data displayed 205 by the client device 110 (as well as to the determined reading behavior in some embodiments) to select 235 the supplemental content. For example, the online medical system 140 generates a prompt including the determined sentiment and the identifier of at least a portion of the data presented by the client device 110 (and including the determined reading behavior in some embodiments). The prompt includes the data displayed 205 by the client device 110 rather than an identifier of the data displayed 205 by the client device 110 in various embodiments. The prompt also includes an instruction to obtain supplemental content based on the determined sentiment (and based on the determined reading behavior in some embodiments). Based on the prompt and relationships learned during a pre-training process, the generative model selects 235 supplemental content. In some embodiments, the generative model generates the supplemental content, while in other embodiments, the generative model determines identifiers of portions of content.

The online medical system 140 transmits 240 the supplemental content to the client device 110, which displays 245 the supplemental content. In various embodiments, the online medical system 140 transmits 240 a prompt including the supplemental content to the client device 110, which displays 245 the prompt. The prompt may be overlaid on a portion of the previously displayed 205 data in various embodiments. In various embodiments, the prompt is configured to receive input from the user, with input received via the prompt transmitted to the online medical system 140, which generates additional content based on the input. The additional content is subsequently transmitted to the client device 110 for display via the prompt. For example, the online medical system 140 applies the generative model to the input received from the user 105 via the prompt and transmits an output of the generative model in response to the input received from the user 105 to the client device 110 as additional content for display via the prompt. Hence, the supplemental content acts as a chatbot with which the user 105 may interact to obtain additional content in various embodiments.

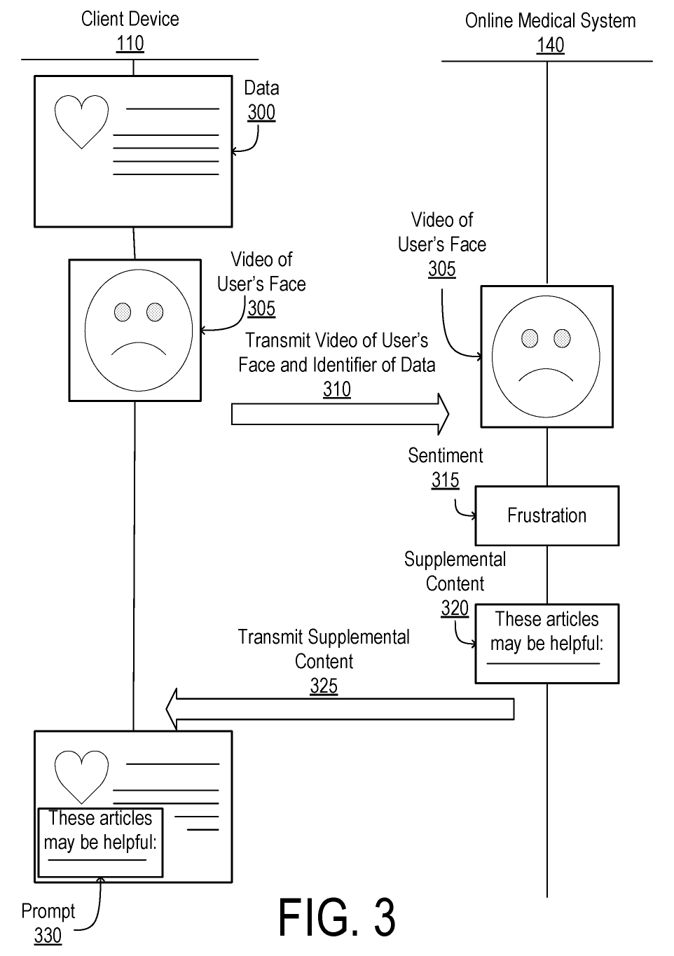

FIG. 3 is a process flow diagram of an example embodiment of a method for selecting supplemental content for a user based on interaction with data captured by a client device 110. As further described above in conjunction with FIGS. 1 and 2, a client device 110 displays data 300 via a medical information application 116 associated with an online medical system 140. The client device 110 may obtain the data 300 from the online medical system 140 or may be obtained from a third-party system 120 in various embodiments.

While displaying the data 300, the medical information application 116 captures interaction by a user 105 of the client device 110 with the data 300. As further described above in conjunction with FIGS. 1 and 2, the interaction includes video 305 of a face of the user 105 viewing the data 300 via the client device 110. For example, the medical information application 116 activates an image capture device 114 of the client device 110 facing in a common direction as the display device 112 of the client device 110 and obtains video captured by the image capture device 114 including the user’s face. The medical information application 116 may capture additional interaction by the user with the data 300, such as inputs for navigating through portions of the data 300 received by the client device 110.

The client device 110 transmits 310 an identifier of the data 300 and the video 305 of the user’s face to an online medical system 140. As further described above in conjunction with FIGS. 1 and 2, the online medical system 140 determines a sentiment 315 of the user 105 from the video 305 of the user’s face. For example, the online medical system 140 applies one or more sentiment analysis models to the video 305 of the user’s face that determine the sentiment 315 of the user based on attributes of the user’s face in the video 305. In the example of FIG. 3, the online medical system 140 determines a sentiment 315 of “frustration” for the user 105 based on the video 305 of the user’s face. As further described above in conjunction with FIGS. 1 and 2, the online medical system 140 also determines a reading behavior of the user for the data 300 based on tracking the user’s gaze in the video 305 of the user’s face in various embodiments.

Based on the sentiment 315 determined for the user 105, the online medical system 140 determines whether to select supplemental content for the user 105. In various embodiments, the online medical system 140 determines to select supplemental content for the user 105 in response to the determined sentiment 315 satisfying one or more sentiment criteria. For example, the online medical system 140 determines to select supplemental content 320 for the user 105 in response to the determined sentiment 315 being included in a set of sentiments. Further, the online medical system 140 may determine to select supplemental content 320 for the user 105 in response to a reading behavior determined for the user 105 satisfying one or more reading criteria, such as in response to the reading behavior determined for the user 105 being in a set of reading behaviors.

In the example of FIG. 3, the online medical system 140 determines to select supplemental content 320 for the user 105 in response to the sentiment 315 of “frustration.” For example, the sentiment of “frustration” is included in a set of sentiments maintained by the online medical system 140. Based on the data 300 displayed by the client device 110 and the sentiment 315 determined for the user 105, the online medical system 140 selects supplemental content 320. In some embodiments, the online medical system 140 selects supplemental content 320 associated with a combination of the identifier of the data 300 and the determined sentiment 315. Alternatively, the online medical system 140 applies a generative model, such as an LLM, to the identifier of the data 300 and to the sentiment 315, with the generative model generating or selecting supplemental content 320 based on the identifier of the data 300 and the sentiment 315. In some embodiments, the online medical system 140 selects supplemental content 320 based on an identifier of the data 300, the determined sentiment, and a determined reading behavior for the user 105; for example, the online medical system 140 stores associations between supplemental content and various combinations of identifiers of data 300, sentiments, and reading behaviors. Further, in various embodiments, the online medical system 140 detemrines a portion of the data 300 the user is viewing based on tracking the user’s gaze from the video 305 of the user’s face and selects 320 supplemental content based on the determined portion of the data the user 105 is viewing and the sentiment 315 (as well as a reading behavior in some embodiments), tailoring the supplemental content 320 to a portion of the displayed data where the user’s gaze was directed when the sentiment 315 was determined.

The online medical system 140 transmits 325 the selected supplemental content 320 to the client device 110, which displays the supplemental content 320. In the example of FIG. 3, the client device 110 displays the supplemental content 320 as a prompt 330 overlaid on a portion of the data 300. This displays the supplemental content 320 in conjunction with the data 300. In various embodiments, the prompt 330 is configured to receive input from the user 105. Input received through the prompt 330 is transmitted to the online medical system 140 in various embodiments, with the online medical system 140 determining additional content based on the input. For example, the online medical system 140 applies a generative model, such as the LLM, to the input (as well as to content previously presented via the prompt 330), and the generative model determines additional content based on the input. The online medical system 140 transmits the additional content to the client device 110, which displays the additional content. For example, the client device 110 displays the additional content in the prompt 330.

The described embodiments incorporate multiple technical improvements to the functioning of computer systems and to computer-based user interfaces, as well as to other technologies and technical fields. For example, the disclosed embodiments simplify interaction with client devices by leveraging video data of a user’s face captured by an image capture device of the client device to determine the user’s sentiment towards data displayed by the client device. The disclosed embodiments leverage the determined sentiment of the user to dynamically select supplemental content for presentation to the user by the client device. This augments data displayed by the client device with supplemental content most applicable to the determined sentiment of the user based on captured video data of the user’s face rather than based on specific interactions the user performs with the client device. Leveraging video data captured by the client device’s image capture device to determine the user’s sentiment towards the displayed data (e.g., frustration, interest, etc.) simplifies selection and display of supplemental content likely to be relevant to the user. Determining the user’s sentiment from captured video data and selecting supplemental content based on previously displayed content and the user’s sentiment reduces an amount of interaction by the user with the client device to obtain or to identify supplemental content relevant to the displayed data, conserving computing resources of the client device. Further, many client devices, such as mobile devices, have display devices with limited display area, so selecting supplemental content based on the user’s determined sentiment more efficiently allocates the display area of a display device for supplemental content more likely to be relevant to the user.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may include a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may include architectures employing multiple processor designs for increased computing capability.

As used herein, unless expressly stated to the contrary, “or” refers to an inclusive “or” and not to an exclusive “or.” For example, a condition “A or B” is satisfied by any one of the following: A is true (or present) and B is false (or not present); A is false (or not present) and B is true (or present); and both A and B are true (or present). Similarly, a condition “A, B, or C” is satisfied by any combination of A, B, and C being true (or present). As a non-limiting example, the condition “A, B, or C” is satisfied when A and B are true (or present) and C is false (or not present). Similarly, as another non-limiting example, the condition “A, B, or C” is satisfied when A is true (or present) and B and C are false (or not present).

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

What is claimed is:

1. A method for supplementing data displayed to a user via a client device, the method comprising:
receiving, at an online medical system, an identifier of data from the online medical system displayed by the client device to the user and interaction by the user with the data, the interaction including video captured by the client device of a face of the user viewing the data via the client device;
determining a sentiment of the user based on one or more attributes of the face of the user included in the captured video;
responsive to determining to present supplemental content to augment the displayed data in response to the sentiment of the user satisfying one or more criteria, selecting supplemental content for presentation to the user based on the sentiment of the user and the data from the online medical system displayed by the client device; and
transmitting the selected supplemental content from the online medical system to the client device.

2. The method of claim 1, wherein transmitting the selected supplemental content from the online medical system to the client device comprises:
transmitting a prompt including the selected supplemental content from the online medical system to the client device.

3. The method of claim 2, wherein the prompt is configured to receive input from the user via the client device.

4. The method of claim 3, further comprising:
generating, by the online medical system, additional content based on input from the user via the prompt; and
transmitting the additional content from the online medical system to the client device.

5. The method of claim 1, wherein selecting supplemental content for presentation to the user based on the sentiment of the user and the data from the online medical system displayed by the client device comprises:
selecting supplemental content including clarification or explanation of one or more portions of the data from the online medical system displayed by the client device in response to determining the sentiment is confusion.

6. The method of claim 1, wherein selecting supplemental content for presentation to the user based on the sentiment of the user and the data from the online medical system displayed by the client device comprises:
selecting supplemental content including a message to pause viewing the data from the online medical system displayed by the client device in response to determining the sentiment is frustration.

7. The method of claim 1, wherein selecting supplemental content for presentation to the user based on the sentiment of the user and the data from the online medical system displayed by the client device comprises:
selecting supplemental content including additional content relating to the data from the online medical system displayed by the client device in response to determining the sentiment is interest.

8. The method of claim 1, wherein selecting supplemental content for presentation to the user based on the sentiment of the user and the data from the online medical system displayed by the client device comprises:
determining a portion of the data from the online medical system displayed by the client device the user is viewing based on a gaze of the user determined from eyes of the user in the captured video; and
selecting supplemental content based on the determined portion of the data from the online medical system displayed by the client device the user is viewing and the sentiment.

9. The method of claim 1, wherein determining the sentiment of the user based on one or more attributes of the face of the user included in the captured video comprises:
determining the sentiment of the user based on one or more attributes of the face of the user included in the captured video; and
determining a reading behavior of the user based on tracking a gaze of the user from eyes of the user in the captured video.

10. The method of claim 9, wherein responsive to determining to present supplemental content to augment the displayed data in response to the sentiment of the user satisfying one or more criteria, selecting supplemental content for presentation to the user based on the sentiment of the user and the data from the online medical system displayed by the client device comprises:
selecting supplemental content for presentation to the user based on the sentiment of the user and the data from the online medical system displayed by the client device in response to determining the sentiment of the user satisfies one or more criteria or in response to determining the reading behavior of the user satisfies one or more criteria.

11. A non-transitory computer readable storage medium having instructions encoded thereon for supplementing data displayed to a user via a client device, the instructions, when executed by one or more processors, cause the one or more processors to perform steps comprising:
receiving, at an online medical system, an identifier of data from the online medical system displayed by the client device to the user and interaction by the user with the data, the interaction including video captured by the client device of a face of the user viewing the data via the client device;
determining a sentiment of the user based on one or more attributes of the face of the user included in the captured video;
responsive to determining to present supplemental content to augment the displayed data in response to the sentiment of the user satisfying one or more criteria, selecting supplemental content for presentation to the user based on the sentiment of the user and the data from the online medical system displayed by the client device; and
transmitting the selected supplemental content from the online medical system to the client device.

12. The non-transitory computer readable storage medium of claim 11, wherein transmitting the selected supplemental content from the online medical system to the client device comprises:
transmitting a prompt including the selected supplemental content from the online medical system to the client device.

13. The non-transitory computer readable storage medium of claim 12, wherein the prompt is configured to receive input from the user via the client device.

14. The non-transitory computer readable storage medium of claim 13 further having instructions encoded thereon that, when executed by the one or more processors, cause the one or more processors to perform steps comprising:
generating, by the online medical system, additional content based on input from the user via the prompt; and
transmitting the additional content from the online medical system to the client device.

15. The non-transitory computer readable storage medium of claim 11, wherein selecting supplemental content for presentation to the user based on the sentiment of the user and the data from the online medical system displayed by the client device comprises:
selecting supplemental content including clarification or explanation of one or more portions of the data from the online medical system displayed by the client device in response to determining the sentiment is confusion.

16. The non-transitory computer readable storage medium of claim 11, wherein selecting supplemental content for presentation to the user based on the sentiment of the user and the data from the online medical system displayed by the client device comprises:
selecting supplemental content including a message to pause viewing the data from the online medical system displayed by the client device in response to determining the sentiment is frustration.

17. The non-transitory computer readable storage medium of claim 11, wherein selecting supplemental content for presentation to the user based on the sentiment of the user and the data from the online medical system displayed by the client device comprises:
selecting supplemental content including additional content relating to the data from the online medical system displayed by the client device in response to determining the sentiment is interest.

18. The non-transitory computer readable storage medium of claim 11, wherein selecting supplemental content for presentation to the user based on the sentiment of the user and the data from the online medical system displayed by the client device comprises:
determining a portion of the data from the online medical system displayed by the client device the user is viewing based on a gaze of the user determined from eyes of the user in the captured video; and
selecting supplemental content based on the determined portion of the data from the online medical system displayed by the client device the user is viewing and the sentiment.

19. The non-transitory computer readable storage medium of claim 11, wherein determining the sentiment of the user based on one or more attributes of the face of the user included in the captured video comprises:
determining the sentiment of the user based on one or more attributes of the face of the user included in the captured video; and
determining a reading behavior of the user based on tracking a gaze of the user from eyes of the user in the captured video.

20. The non-transitory computer readable storage medium of claim 19, wherein responsive to determining to present supplemental content to augment the displayed data in response to the sentiment of the user satisfying one or more criteria, selecting supplemental content for presentation to the user based on the sentiment of the user and the data from the online medical system displayed by the client device comprises:
selecting supplemental content for presentation to the user based on the sentiment of the user and the data from the online medical system displayed by the client device in response to determining the sentiment of the user satisfies one or more criteria or in response to determining the reading behavior of the user satisfies one or more criteria.
